(19) United States
(12) Patent Application Publication
(10) Pub. No.: US 2022/0134094 A1
(43) Pub. Date: May 5, 2022

(54) SKIN CARE DEVICE

(71) Applicant: K CO., LTD., Hwaseong-si, (KR)

(72) Inventors: Jung Rack KIM, Hwaseong-si (KR); Dong Hoo KIM, Osan-si (KR); Ji An KIM, Gwacheon-si (KR)

(73) Assignee: K CO., LTD., Hwaseong-si (KR)

(21) Appl. No.: 17/512,630

(22) Filed: Oct. 27, 2021

(30) Foreign Application Priority Data
Oct. 29, 2020 (KR) 10-2020-0141904

(51) Int. Cl. A61N 1/32; A61N 1/06; A61H 15/00; A61H 23/02

(52) U.S. Cl. CPC A61N 1/328; A61N 1/06; A61H 2201/0153; A61H 23/0254; A61H 2201/1692; A61H 15/0085

(57) ABSTRACT

The present invention is to solve the problems of prior arts and to obtain a more effective skin beauty effect, in particular, physical massage and electrical stimulation at the same time on both sides along the face line. It is to provide a skin care device that is more suitable for skin care for the face, such as providing a more effective skin improvement effect by providing. The skin care device according to an embodiment of the present invention for this purpose, a handle provided with a first pressurizing supporter and a second pressurizing supporter; A first massager provided at an end of the first pressurizing supporter and having a pair of massage rollers; A second massager provided at an end of the second pressurizing supporter and having a pair of massage rollers; and a controller that provides radio-frequency electrical stimulation.

### BACKGROUND OF THE INVENTION

### Field of the Invention

The present invention relates to a skin care device, and more specifically, the skin care device according to the present invention prevents skin aging and improves skin elasticity by simultaneously providing massage and radio-frequency electrical stimulation to the skin of the face, body, etc.

### Description of the Related Art

In general, as a person's skin ages, elasticity decreases and the skin stretches and wrinkles, and when exposed to UV light for a long time, the skin becomes thicker and the skin's blood circulation ability decreases, and moisture and nutrients are not supplied smoothly to the skin.

In order to solve this problem, a functional substance such as a nourishing cream has been applied to a skin, or a skin massage method for providing a physical stimulation to the skin has been used to further facilitate blood circulation.

In addition, it is already known that the radio-frequency electric stimulation of the skin promotes absorption of nutrients applied to the skin and promotes blood circulation by generating deep heat in the skin.

A number of prior art documents related to a skin beauty device to simultaneously provide massage and radio-frequency electric stimulation for the skin have been published, Korean patent publications No. 10-2020-0101170, No. 10-2017-0068697, No. 10-2016-0086275.

Prior arts as described above are provided with a plurality of electrodes on the surface in contact with the skin to provide high-frequency electricity to the contact surface of the skin through the electrodes to promote absorption of creams and drugs applied to the skin, and deep heat to the skin can promote blood circulation.

However, when high-frequency electrical stimulation is applied to the skin through electrodes provided on the surface in contact with the skin, there is a risk of burns due to the heat generated by the energy of the high-frequency electricity. In addition, it is difficult to increase the effect of skin beauty by simply applying electrical stimulation to the skin.

### SUMMARY OF THE INVENTION

The present invention is to solve the problems of the prior arts as described above and to achieve a more effective skin beauty effect, in particular, by providing high-frequency electricity to the skin and simultaneously tapping the skin while rubbing the skin to promote absorption of a cream or drug applied to the skin and generate deep heat in the skin to promote blood circulation. The present invention is to provide a skin care device capable of greatly improving the effect of skin care by providing a complex massage of rubbing and tapping on the skin while preventing skin burns due to heat of an electrode by high-frequency electricity.

In accordance with an aspect of the present invention, the above and other objects can be accomplished by the provision of a skin care device that delivers stimulation for skin care by contacting the skin, comprising a handpiece provided for the user to grip, a signal generator that generates a high frequency electric signal to provide high frequency electricity to the contact surface of the skin, and a head provided at a distal end of the handpiece to be in contact with the skin, wherein the head rubs and taps the contact surface of the skin while providing high-frequency electricity to the contact surface of the skin by the high-frequency electric signal of the signal generator.

Preferably, the head is configured to perform an operation of rubbing the contact surface of the skin by eccentric rotation by driving force of a driving motor inside the handpiece at the distal end of the handpiece.

Preferably, a vibration module is provided in the head to perform an operation of tapping the contact surface of the skin by vibrating the head by the vibration module.

Preferably, the skin care device further comprises a driving motor provided in the handpiece, and a driving shaft rotating by the driving motor and penetrating the distal end of the handpiece, wherein the head is rotatably coupled to the distal end of the handpiece, and the driving shaft is fixed to the eccentric position of the head, such that the head rotates eccentrically and rubs the contact surface of the skin as the driving shaft rotates by the driving motor.

Preferably, the head comprises a head body, an eccentric neck provided at an eccentric position from the center of the head body and rotatably coupled to the distal end of the handpiece to support the eccentric rotation of the head, and a head electrode provided at an end of the head body and having a surface contacting the skin, wherein the head electrode includes electrodes that receives a high-frequency electric signal from the signal generator and provides high-frequency electricity to a contact surface of the skin.

Preferably, the head further includes a head frame configured to fix the head electrode to the head body, and wherein the head frame includes an eccentric shaft fixing part provided at an eccentric position corresponding to the eccentric neck to fix the end of the driving shaft of the driving motor, whereby the head rotates eccentrically around the eccentric shaft fixing part to which the drive shaft is fixed and the eccentric neck as the drive shaft rotates by the drive motor.

Preferably, the skin care device further comprises a head frame for fixing the head electrode to the head body, a head substrate fixed to the head frame so as to transfer a high frequency electric signal of the signal generator to the head electrode, and a vibration module electrically connected to and fixed to the head substrate and transmitting vibration to the head electrode.

Preferably, the head includes a head electrode which forms a surface in contact with the skin and includes a plurality of bipolar electrodes that receive high-frequency electric signals from the signal generator and provide high-frequency electricity to the contact surface of the skin, and a head frame including an eccentric shaft fixing part provided at an eccentric position from the center of the head frame to fix the end of a driving shaft of a driving motor provided inside the handpiece, wherein the head frame fixes the plurality of bipolar electrodes constituting the head electrode so that the plurality of bipolar electrodes can be in contact with the skin, whereby the head rotates eccentrically around the eccentric shaft fixing part to which the driving shaft is fixed, and thus the plurality of bipolar electrodes of the head electrode provide high-frequency electricity to the contact surface of the skin as the driving shaft rotates by the driving motor.

Preferably, the head includes a head electrode including a plurality of circular electrodes provided to form concentric circles having a surface being in contact with the skin, wherein the plurality of circular electrodes receive high-frequency electric signals from the signal generator and provide high-frequency electricity to a contact surface of the skin, a head frame fixing the plurality of circular electrodes thereto, and a head substrate fixed to the head frame and forming a plurality of circular electrode patterns corresponding to the plurality of circular electrodes so as to transmit high-frequency electric signal from the signal generator to the head electrode.

### BRIEF DESCRIPTION OF THE DRAWINGS

FIG. 1 shows perspective views (a) and (b) of the skin care device according to an embodiment of the present invention viewed from different perspectives.

FIG. 2 shows a block diagram for describing a control system of a skin care device according to an embodiment of the present invention.

FIG. 3 shows a side cross-sectional view of a skin care device according to an embodiment of the present invention.

FIG. 4 shows an internal configuration by translucent processing a handpiece of the skin care device.

FIG. 5 shows a view (a) separately shown with respect to the driving shaft and the head in the configuration shown in FIG. 4, and an exploded view (b) with respect to the driving shaft and the head shown in FIG. 4(a).

FIG. 6 shows a view (a) viewed from the head electrode of the head provided in the handpiece of the skin care device according to an embodiment of the present invention, and views (b) to (f) illustrating eccentric rotation of the head, respectively.

### DESCRIPTION OF THE PREFERRED EMBODIMENTS

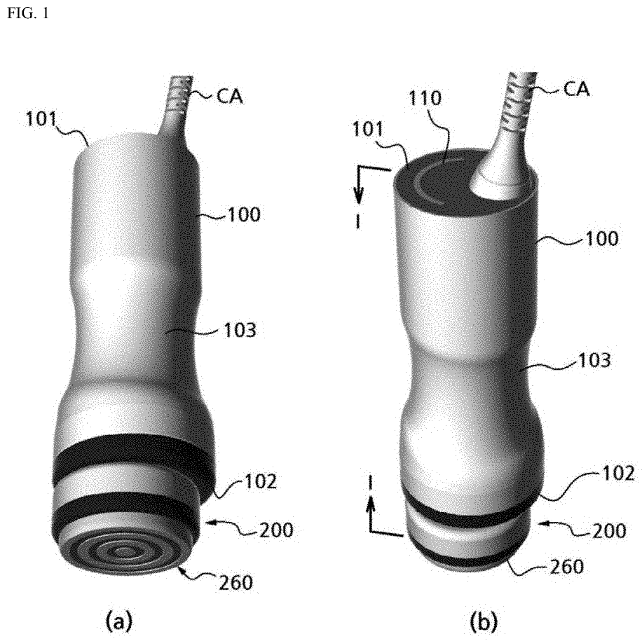

As shown in FIG. 1, the skin care device according to an embodiment of the present invention includes

Hereinafter, specific details of the skin care device according to the present invention will be described with reference to the drawings.

First, an overall configuration of a skin care device according to an embodiment of the present invention will be described with reference to FIG. 1. FIGS. 1(a) and (b) respectively show a perspective view of the skin care device according to an embodiment of the present invention viewed from different perspectives.

As shown in FIGS. 1(a) and (b), the skin care device according to an embodiment of the present invention is provided with a handpiece 100 provided so that the user can grip.

The handpiece 100 has a proximal end 101 and a distal end 102 as shown in FIG. 1. A grip portion 103 concavely formed so that a user can easily grip it.

An LED module 110 is provided at the proximal end 101 of the handpiece 100 to display the operating state or abnormality of the skin care device. For example, when the power is connected to the device but the operation is stopped, the LED module 110 may display a red light, and may display a blue light during the device operating. For example, when the temperature of the electrode of a head, which will be described later, approaches a set temperature, the LED module 110 may display flickering.

The distal end 102 of the handpiece 100 is provided with a head 200 that delivers stimulation for beauty in contact with the skin, and a signal generator (not shown) electrically connected to the handpiece 100 may be provided.

The signal generator (not shown) may be provided outside the handpiece 100, may be electrically connected to the handpiece 100 by a cable CA, and may generate a high-frequency electrical signal to apply high-frequency electrical stimulation to the skin for cosmetic purposes.

The handpiece 100 may be configured to be electrically connected to the main body (not shown) of the device, and the signal generator may be provided in the main body. The main body is provided with a power supply device in addition to the signal generator. Power can be supplied from the main body to the handpiece through a cable CA. A controller (not shown) may be provided in the main body, and the controller may control generation of a high-frequency electrical signal from the signal generator, and may control operation of the head.

The controller as described above may be mounted inside the handpiece rather than the main body.

The high-frequency electrical signal generated by the signal generator is transmitted to the handpiece 100 through a cable CA, and the electrical signal of the signal generator may be transmitted to the head 200 through an electrical connection means (not shown) connected from the proximal end 101 to the distal end 102.

The head 200 provided on the distal end 102 of the handpiece, has a surface in contact with the skin, and can perform various operations for skin care by receiving a high-frequency electrical signal from the signal generator.

A surface of the head 200 in contact with the skin is provided with a head electrode 260, and high-frequency electricity from the signal generator can be transmitted to the skin through the head electrode 260.

When high-frequency electrical stimulation is applied to the skin, deep heat is generated in the skin, which promotes blood circulation in the skin. In addition, when high-frequency electrical stimulation is applied in a state in which a cream or liquid is applied to the skin, the applied cream or liquid is rapidly absorbed into the dermal layer of the skin.

However, since heat is generated by the energy of high-frequency electricity when high-frequency electrical stimulation is applied to the skin through the head electrode 260 provided on the surface in contact with the skin, the heat of the head electrode 260 causes the skin to burn.

In the skin care device according to an embodiment of the present invention, the head 200 may rub the contact surface of the skin to prevent the risk of burns to the skin while generating deep heat on the skin by applying high-frequency electrical stimulation to the skin through the head electrode 260.

Since the head 200 performs a skin massage effect while rubbing against the skin, physical stimulation may be applied to the skin contact surface to facilitate blood circulation and discharge of waste products, thereby significantly improving the effect of skin beauty.

The operation of the head 200 rubbing the contact surface of the skin may be implemented by eccentric rotation of the head 200 by the driving force of the driving motor inside the handpiece 100, and the operation of the head 200 tapping the contact surface of the skin may be implemented by vibration of the head.

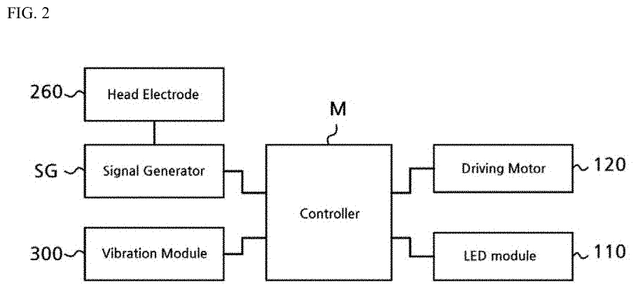

Meanwhile, a control system of a skin care device according to an embodiment of the present invention will be described with reference to the block diagram of FIG. 2.

In the skin care device according to an embodiment of the present invention, each component is controlled by a controller M provided in a main body or a handpiece, and the components controlled by the controller M may include a signal generator SG, an LED module 110, a driving motor 120, and a vibration module 300.

The signal generator (SG) is a device for generating a high frequency electric signal transmitted to the head electrode 260 provided on a surface contacting the skin of the head provided at the end of the handpiece, for example, the signal generator (SG) may generate a high frequency electric signal by a user pressing a finger button or a foot button. At this time, the controller (M) may control the signal generator (SG) to generate a high frequency electrical signal according to preset conditions for output power, pulse, application time, or conditions set by the user.

The controller M may determine whether the skin care device is currently operating and control the LED module 110 to change the color emitted or to change the emission pattern according to the operating state.

A driving motor 120 rotates the driving shaft so that the head 200 can rotate eccentrically, and the controller M may control the rotation speed, rotation direction, operation time, etc. of the driving motor 120 according to a preset condition or a condition set by the user.

The vibration module 300 operates by a user's foot-switch operation to vibrate the head, and in this case, the controller M may control the intensity of vibration, vibration period, vibration pattern, etc. generated by the vibration module 300 according to a preset condition or a condition set by the user.

According to the control system described above, when the user grips the handpiece to press the foot switch while contacting the head electrode of the head to operate the device, the controller M controls the signal generator SG to generate a high frequency electric signal under a set condition, and the high frequency electric signal is transmitted to the contact surface of the skin.

In addition, the controller M may control the driving motor 120 to control the head to eccentrically rotate while the head electrode 260 is in contact with the skin so that the head rubs the contact surface of the skin, and the controller M controls the vibration module 300 to vibrate according to a set vibration condition.

Meanwhile, a temperature sensor (not shown) may be mounted on the head, and the temperature sensor measures the temperature of the head electrode or the temperature of the contact surface of the skin and transmits the measured temperature to the controller, hence controlling the signal generator or power supply device to prevent skin burns.

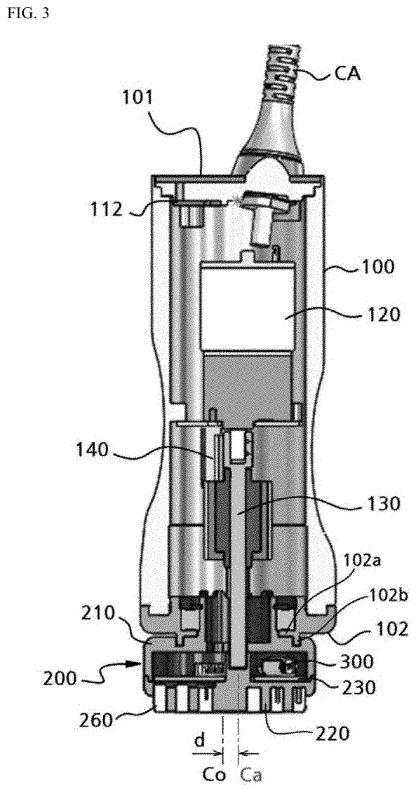

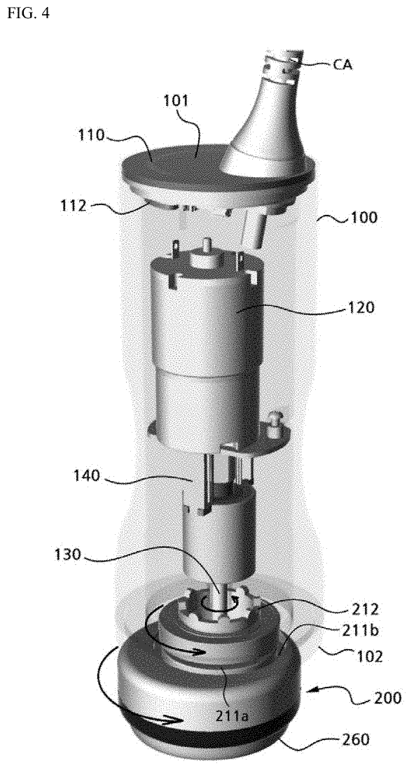

Meanwhile, a configuration inside the skin care device according to an embodiment of the present invention will be described with reference to FIGS. 3 and 4.

FIG. 3 is a side cross-sectional view of a skin care device according to an embodiment of the present invention, and FIG. 4 is a view showing an internal configuration by translucent processing a handpiece of the skin care device.

As shown in FIGS. 3 and 4, in the skin care device according to an embodiment of the present invention, a cable CA is connected to a proximal end 101 of the handpiece 100, and a power line of the cable CA is connected to a head 200 inside the handpiece 100.

The power line of the cable CA may also be connected to the LED substrate 112 provided inside the proximal end 101 of the handpiece 100, so that the LED substrate 112 controls the operation of the LED module 110.

As shown in FIGS. 3 and 4, a driving motor 120 may be provided inside the handpiece 100, the driving motor 120 may rotate the driving shaft 130, and the driving shaft 130 may be guided by a driving shaft guide 140 provided between the driving motor 120 and the distal end 102. The drive shaft 130 passes through the distal end 102 from the drive motor 120 and is fixed to the head 200.

More specifically, the drive shaft 130 penetrating the distal end 102 of the handpiece is fixed to a predetermined eccentric position from the center of the head 200, and when the driving motor 120 rotates the drive shaft 130, the head 200 fixed to the drive shaft 130 rotates by the drive shaft 130, but since the drive shaft 130 rotates at an eccentric position from the center of the head 200, the head 200 rotates eccentrically.

As shown in FIGS. 3 and 4, the position at which the driving shaft 130 is fixed to the head is an eccentric position from the center of the head in the vertical direction.

Specifically, as shown in FIG. 3, when Ca indicates the center line of the driving shaft 130 and Co indicates the center line of the head 200, the center line Ca of the drive shaft 130 is different from the center line Co of the head 200 by a distance d, and the drive shaft 130 is eccentric with respect to the head 200 by the difference.

Meanwhile, as shown in FIG. 3, the head 200 may include a head body 210, a head frame 220, a head substrate 230, a head electrode 260, and the like.

As shown in FIGS. 3 and 4, the head body 210 is configured to fix the head frame 220 and the head substrate 230, and an eccentric neck 212 provided at a position eccentric from the center Co of the head body 210 is provided on the upper part of the head body 210.

The eccentric neck 212 provided on the upper portion of the head body 210 may be rotatably coupled to penetrate the distal end 102 of the handpiece.

As shown in FIG. 3, as the eccentric neck 212 is coupled to the distal end 102 of the handpiece, the neck supporter 102a of the distal end 102 of the handpiece is accommodated in the support groove 211a formed in the eccentric neck 212.

A state in which the head body 210 is coupled to the distal end 102 of the handpiece may be supported by a configuration in which the neck supporter 102a is accommodated in the support groove 211a, and when the head 200 rotates, the neck supporter 102a may guide rotation of the head 200 along the support groove 211a.

In addition, as shown in FIG. 4, the rotation guide 102b of the distal end 102 of the handpiece as shown in FIG. 3 is accommodated in the guide groove 211b, and when the head 200 rotates, the rotation guide 102b may guide the rotation of the head 200 along the guide groove 211b.

Meanwhile, the head frame 220 provided in the head body 210 fixes the head substrate 230 and the head electrode 260 in the head body 210. The head substrate 230 is configured to have an electrical component and an electrode pattern for implementing the function and operation of the head 200, and transmits a high-frequency electrical signal of the signal generator to the head electrode 260.

The head substrate 230 may be electrically connected to a power line of the cable CA to operate various electrical components mounted on the circuit by receiving power, and a vibration module 300 for vibrating the head 200 may be electrically connected to the head substrate 230 to be fixed on the circuit.

The head electrode 260 is fixed to the head frame 220 and exposed to a surface contacting the skin at the end of the head 200, and the head electrode 260 includes an electrode receiving a high frequency electric signal from the signal generator to provide high frequency electricity to the contact surface of the skin.

The head frame 220 can support the entire head 200 to stably vibrate when the vibration module 300 provided on the head substrate 230 is operated while fixing the head substrate 230 and the head electrode 260.

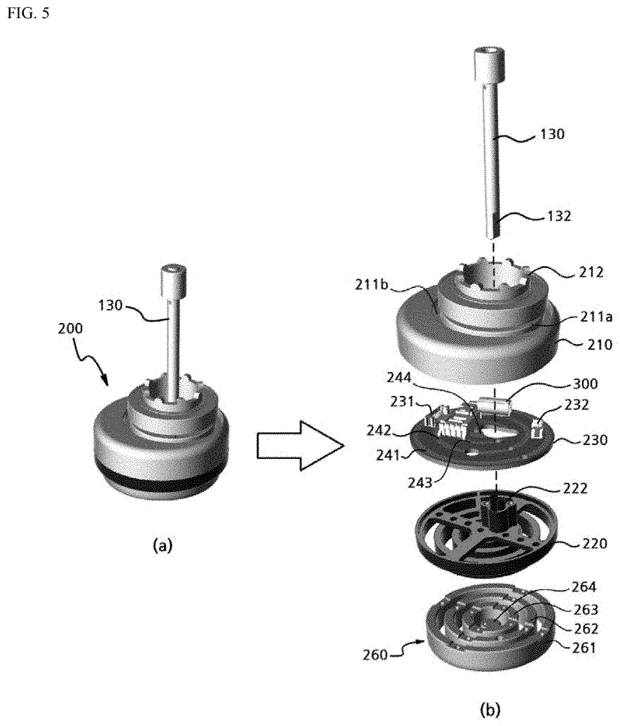

Meanwhile, a coupling structure between the head and the drive shaft of the skin care device according to an embodiment of the present invention and a more specific configuration of the inside of the head will be described with reference to FIG. 5.

FIG. 5(a) is a view separately shown with respect to the driving shaft 130 and the head 200 in the configuration shown in FIG. 4, and FIG. 5(b) is an exploded view with respect to the driving shaft 130 and the head 200 shown in FIG. 4(a).

As shown in FIG. 5(b), the head 200 may include the head body 210, the head substrate 230, the head frame 220, and the head electrode 260, and the driving shaft 130 may be fixed to the head frame 220 through the head body 210 and the head substrate 230.

An eccentric neck 212 may be provided at an eccentric position from the center of the head body 210 and rotatably coupled to the distal end of the handpiece to support the eccentric rotation of the head 200.

The neck supporter 102a of the distal end 102 of the handpiece may be accommodated in the support groove 211a formed in the eccentric neck 212, and the rotation guide 102b of the distal end 102 of the handpiece may be accommodated in the guide groove 211b formed in the head body 210, so that the eccentric neck 212 may rotate at the distal end 102 of the handpiece as the head 200 rotates.

The eccentric neck 212 may be hollow so that the drive shaft 130 may pass therethrough, and the shaft end part 132 of the drive shaft 130 penetrating the hollow inside of the eccentric neck 212 may be fixed to the eccentric shaft fixing part 222 provided at an eccentric position from the center of the head frame 220.

As described above, as the shaft end part 132 is fixed to the eccentric shaft fixing part 222 of the head frame 220, when the driving shaft 130 is rotated by the driving motor, the head 200 is rotated by the driving shaft 130. Since the drive shaft 130 is eccentric from the center of the head 200, the head 200 rotates eccentrically according to the rotation of the drive shaft 130.

Meanwhile, the head electrode 260 may be fixed to the head frame 220, and as shown in FIG. 5(b), a plurality of circular electrodes 261, 262, 263, and 264 constituting the head electrode 260 may be fixed to the head frame 220, respectively.

The plurality of circular electrodes 261, 262, 263, and 264 are concentrically formed as shown in FIG. 5(b), and form a surface where lower ends of the plurality of circular electrodes contact the skin.

It is preferable that the plurality of circular electrodes 261, 262, 263, and 264 are formed in bipolar electrodes.

For example, in the plurality of circular electrodes 261, 262, 263, and 264 as shown in FIG. 5(b), the circular electrode 261 and the circular electrode 262 may be configured as a pair of bipolar electrodes, and the circular electrode 263 and the circular electrode 264 may be configured as a pair of bipolar electrodes.

Each of the plurality of circular electrodes 261, 262, 263, and 264 may be configured as a monopolar electrode, but in this case, a separate ground electrode is required, and since the monopolar electrode type is mainly used in a surgical high-frequency electrode such as ablation of skin tissue, bipolar electrodes type may be more suitable for the skin care device that transmits electrical stimulation.

Meanwhile, as shown in FIG. 5(b), the head substrate 230 may be coupled and fixed to the head frame 220, and the head substrate 230 may include signal connectors 231 and 232 to input a high frequency electrical signal transmitted from the signal generator, and may be electrically connected from a cable of the handpiece to a signal connection line.

The signal connector may include a first signal connector 231 for inputting (+) electrode signal and a second signal connector 232 for inputting (−) electrode signal.

The first signal connector 231 may be connected to a (+) circular electrode, and the second signal connector 232 may be connected to a (−) circular electrode such that the (+) circular electrode and the (−) circular electrode form a pair of bipolar electrodes.

As shown in FIG. 5(b), circular electrode patterns 241, 242, 243, and 244 may be formed on the head substrate 230 at positions corresponding to each of the circular electrodes 261, 262, 263, and 264, and each of the circular electrode patterns 241, 242, 243, and 244 may be configured to electrically contact or connected to each of the circular electrodes 261, 262, 263, and 264 at corresponding positions.

For example, the circular electrode patterns may be formed to correspond to the circular electrodes, and thus each of the circular electrode patterns may be electrically connected to each corresponding circular electrode by electrically direct contact with each other (or through a conductive mediating member).

For another example, a spring-loaded pin (Pogo-Pin) may be used to connect between the circular electrode pattern and the corresponding circular electrode. One end of the Pogo-Pin may be connected and fixed to the circular electrode pattern, and the other end (e.g., a pin part that can be elastically slid by a spring) of the Pogo-Pin may electrically contact the circular electrode.

According to the above-described configuration, in the skin care device according to an embodiment of the present invention, the signal generator may be electrically connected to the signal connectors 231 and 232 by the cable, and the signal connectors 231 and 232 may be connected to the circular electrodes 261, 262, 263 and 264 through circular electrode patterns 241, 242, 243, and 244.

Accordingly, the head 200 of the skin care device according to an embodiment of the present invention may transmit high-frequency electrical stimulation to the skin through the head electrode 260 as the high frequency electrical signal generated by the signal generator may be input to the signal connectors 231 and 232 to be transmitted to the head substrate 230 and transferred to the circular electrodes 261, 262, 263, and 264 through the circular electrode patterns 241, 242, 243, and 244 on the head substrate 230.

As shown in FIG. 5(b), as the head electrode 260 is configured as bipolar electrodes, (+) electrode signal is input to the first signal connector 231 and transmitted to the (+) circular electrodes (e.g., 261 and 263) through (+) circular electrode patterns (e.g., 241 and 243). Then, (−) electrode signal is input to the second signal connector 232 and transmitted to the (−) circular electrodes (e.g., 262 and 264) through (−) circular electrode patterns (e.g., 242 and 244). Accordingly, the (+) circular electrodes (e.g., 261 and 263) and the (−) circular electrodes (e.g., 262 and 264) may transmit high frequency electrical stimulation as the bipolar electrodes pair.

Meanwhile, as shown in FIG. 5(b), the vibration module 300 may be provided on the head substrate 230 to allow the head electrode 260 to tap the skin by vibration.

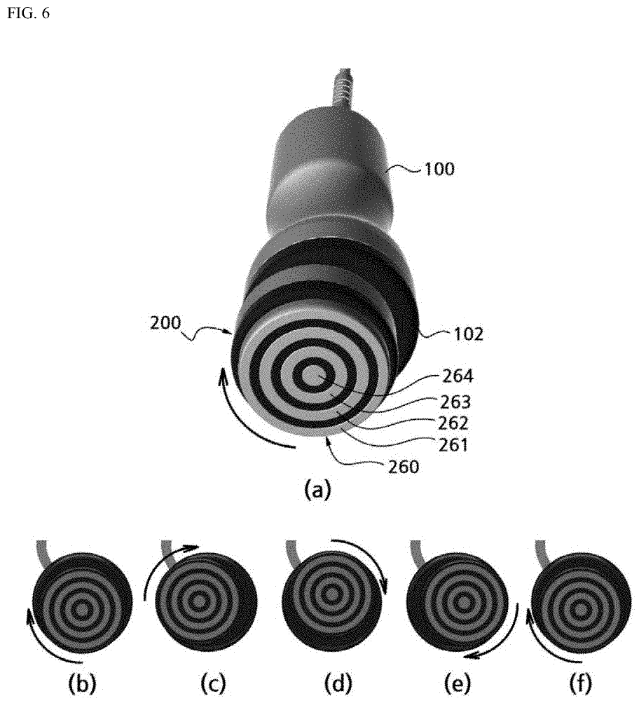

An example in which the skin care device according to an embodiment of the present invention operates according to the above-described configuration is shown in FIG. 6.

FIG. 6(a) shows a view viewed from the head electrode 260 of the head 200 provided in the handpiece 100 of the skin care device according to an embodiment of the present invention, and FIGS. 6(b) to (f) show eccentric rotation of the head, respectively.

As shown in FIG. 5(b) and FIG. 6(a), the head electrode 260 of the head 200 may be provided with a plurality of circular electrodes 261, 262, 263, and 264, and these circular electrodes may be configured such that two adjacent electrodes form a bipolar electrode pair to transfer high frequency electrical stimulation.

In the embodiment shown in FIG. 5(b) and FIG. 6(a), a case where the head electrode 260 has a plurality of circular electrodes 261, 262, 263, and 264 is shown, but the present invention is not limited thereto.

As shown in FIGS. 5 and 6(a), the entire surface of the head 200 in contact with the skin is circular because it is effective that the contact surface is circular when the head performs an eccentric rotation and rubbing the skin.

However, it is not necessary to make the contact surface of the head circular, and it is also possible to be configured as a triangle, a square, or more polygon.

And, as shown in FIGS. 5 and 6, the electrode that transmits high-frequency electrical stimulation to the skin is not limited to a circular electrode, and various types of electrodes can be configured.

For example, multiple straight electrodes may be disposed to form a shape such as “ [symbol] ”, or multiple small square-shaped electrodes may be disposed to form a shape such as “ [symbol] ”. In this case, electrode patterns may be formed on the head substrate corresponding to electrodes (e.g., electrode patterns corresponding to the shape of the electrodes may be formed on the head electrode).

Meanwhile, according to the coupling structure of the driving shaft and the head as described with reference to FIGS. 3 to 5, an example of a case in which the head 200 rotates eccentrically in the direction of the arrow shown in FIG. 6(a) is shown in FIGS. 6(b) to (f).

That is, as the head 200 rotates eccentrically while in contact with the skin, the heads may rotate in the order of (b)→(c)→(d)→(e)→(f) of FIG. 6 to rub the skin.

In this case, as high-frequency electric stimulation is transmitted to the skin through each of the electrodes (261, 262, 263, 264), the head eccentrically rotates to rub the skin, and the head vibrates due to vibration caused by the vibration module.

As described above, the skin care device according to an embodiment of the present invention may provide high-frequency electricity to the skin and tap the skin in order to promote absorption of cream and chemical liquid applied to the skin and generate deep heat to promote blood circulation. Accordingly, the skin care device can greatly improve the effect of skin beauty by providing a complex massage of rubbing and tapping the skin while preventing skin burns due to heat in the electrode providing high frequency electric stimulation.

What is claimed is:

1. A skin care device that delivers stimulation for skin care by contacting the skin, comprising:
a handpiece provided for the user to grip;
a signal generator that generates a high frequency electric signal to provide high frequency electricity to the contact surface of the skin; and
a head provided at a distal end of the handpiece to be in contact with the skin, wherein the head rubs and taps the contact surface of the skin while providing high-frequency electricity to the contact surface of the skin by the high-frequency electric signal of the signal generator.

2. The skin care device according to claim 1, wherein the head is configured to perform an operation of rubbing the contact surface of the skin by eccentric rotation by driving force of a driving motor inside the handpiece at the distal end of the handpiece.

3. The skin care device according to claim 1, wherein a vibration module is provided in the head to perform an operation of tapping the contact surface of the skin by vibrating the head by the vibration module.

4. The skin care device according to claim 1, further comprising a driving motor provided in the handpiece, and a driving shaft rotating by the driving motor and penetrating the distal end of the handpiece, wherein the head is rotatably coupled to the distal end of the handpiece, and the driving shaft is fixed to the eccentric position of the head, such that the head rotates eccentrically and rubs the contact surface of the skin as the driving shaft rotates by the driving motor.

5. The skin care device according to claim 1, wherein the head comprises:
a head body;
an eccentric neck provided at an eccentric position from the center of the head body and rotatably coupled to the distal end of the handpiece to support the eccentric rotation of the head; and
a head electrode provided at an end of the head body and having a surface contacting the skin, wherein the head electrode includes electrodes that receives a high-frequency electric signal from the signal generator and provides high-frequency electricity to a contact surface of the skin.

6. The skin care device according to claim 5, wherein the head further includes a head frame configured to fix the head electrode to the head body, and
wherein the head frame includes an eccentric shaft fixing part provided at an eccentric position corresponding to the eccentric neck to fix the end of the driving shaft of the driving motor,
whereby the head rotates eccentrically around the eccentric shaft fixing part to which the drive shaft is fixed and the eccentric neck as the drive shaft rotates by the drive motor.

7. The skin care device according to claim 5, further comprising:
a head frame for fixing the head electrode to the head body;
a head substrate fixed to the head frame so as to transfer a high frequency electric signal of the signal generator to the head electrode; and
a vibration module electrically connected to and fixed to the head substrate and transmitting vibration to the head electrode.

8. The skin care device according to claim 1, wherein the head includes:
a head electrode which forms a surface in contact with the skin and includes a plurality of bipolar electrodes that receive high-frequency electric signals from the signal generator and provide high-frequency electricity to the contact surface of the skin; and
a head frame including an eccentric shaft fixing part provided at an eccentric position from the center of the head frame to fix the end of a driving shaft of a driving motor provided inside the handpiece, wherein the head frame fixes the plurality of bipolar electrodes constituting the head electrode so that the plurality of bipolar electrodes can be in contact with the skin,
whereby the head rotates eccentrically around the eccentric shaft fixing part to which the driving shaft is fixed, and thus the plurality of bipolar electrodes of the head electrode provide high-frequency electricity to the contact surface of the skin as the driving shaft rotates by the driving motor.

9. The skin care device according to claim 1, wherein the head includes:
a head electrode including a plurality of circular electrodes provided to form concentric circles having a surface being in contact with the skin, wherein the plurality of circular electrodes receive high-frequency electric signals from the signal generator and provide high-frequency electricity to a contact surface of the skin;
a head frame fixing the plurality of circular electrodes thereto; and
a head substrate fixed to the head frame and forming a plurality of circular electrode patterns corresponding to the plurality of circular electrodes so as to transmit high-frequency electric signal from the signal generator to the head electrode.
